(19) United States
(12) Patent Application Publication
(10) Pub. No.: US 2004/0068234 A1
(43) Pub. Date: Apr. 8, 2004

(54) Bone graft particle delivery apparatus and method

(76) Inventors: Jeff Martin, Germantown, TN (US); Joseph Ferrante, Bartlett, TN (US); Anthony James, Bartlett, TN (US); Marc Long, Memphis, TN (US); David Castleman, Bartlett, TN (US); Jeff Schryver, Memphis, TN (US)

Correspondence Address: Chief Patent Counsel, Smith & Nephew, Inc., 1450 Brooks Road, Memphis, TN, 38116, US

(21) Appl. No.: 10/264,140

(22) Filed: Oct. 3, 2002

(51) Int. Cl. A61M 5/00

(52) U.S. Cl. 604/187

(57) ABSTRACT

Embodiments of the present invention include apparatuses and methods for delivering one or more particles. In some embodiments, shaped particles are stacked in a tube and dispensed from the tube into a wound such as a bone void.

### TECHNICAL FIELD

Embodiments of the invention are directed generally to the controlled delivery of particles. More specifically, apparatuses and methods for delivering bone graft particles to a bone repair or wound site are disclosed. In some embodiments, the particles are delivered through a tube that may also be used to store the bone graft particles.

### BACKGROUND OF THE INVENTION

Bone grafting materials have been used for many years to assist with the healing of damaged bones, and to replace structures removed from bones as may occur in a joint replacement surgery. The general principle is that a bone grafting material is placed in a bone void to provide a temporary, replacement structure. In an ideal instance, healthy portions of the bone will grow into and replace the bone grafting material with new, healthy bone. A particularly advantageous type of bone grafting material is the JAX® brand bone graft particle manufactured by Smith & Nephew, Inc. JAX® brand bone graft particles provide a unique shape that promotes interlocking among the particles, and therefore gives greater cohesion and shear strength to a group of particles used to fill a bone void. This and other features and embodiments of bone graft particles, and methods for their manufacture and use, are disclosed in U.S. patent application Ser. No. 09/517,981, SHAPED PARTICLE AND COMPOSITION FOR BONE DEFICIENCY AND METHOD OF MAKING THE PARTICLE; Ser. N0. 09/792,681, MANUFACTURE OF BONE GRAFT SUBSTITUTES; Ser. No. 10/054,523, PACKAGING AND DELIVERY SYSTEM FOR BONE GRAFT PARTICLES; and Ser. No. 10/099,616, SHAPED PARTICLE COMPRISED OF BONE MATERIAL AND METHOD OF MAKING THE PARTICLE, all to Smith & Nephew, Inc. Each of these applications is hereby incorporated by reference in the present application.

The unique interlocking shape of JAX® brand bone graft particles creates a challenge to delivering the particles to a bone void. This is particularly true where a less invasive procedure is being accomplished. Less invasive or “minimally invasive” orthopedic surgery is a goal due to the fact that such procedures typically result in shorter recovery times, less pain, and lower morbidity. With a less invasive procedure, a smaller incision is made. Sometimes the incision will be less than an inch long, and endoscopic, fluoroscopic, or computer assisted equipment and techniques will be used to guide the procedure such that it may be accomplished through the small incision. A favored instrument for delivering bone graft particles through a small incision is a tube or syringe. Bone grafting materials of the prior art are putties, gels, powders, and mixtures of some or all of these. Gels, powders, and some mixtures can be easily extruded or poured from a syringe or tube. However, such materials have very low shear strength. The prior art materials therefore provide very low shear strength and poor performance when in a bone void and are often inadvertently displaced. Putties tend to be denser and less susceptible to being displaced, but fail to provide adequate paths through which bone regrowth can occur. Putties can also be difficult to place through a small incision.

A randomly distributed interlocking mixture (e.g. including JAX®), whether dry or consisting of a mixture of particles and fluids, will not extrude well through a typical syringe. When such a mixture is pushed toward the exit hole of the syringe, the interlocked particles tend to jam against the walls or exit hole of the syringe. Stated another way, if interlocking of particles is allowed to occur such that shear displacements in the planes parallel with the axis of the syringe are restricted, the particles will tend to extrude or flow poorly. Groups of interlocked particles are typically not well suited for delivery from a tube because the particles are bound together and tend to flow poorly. This results in difficulty in placing the particles accurately through a small incision.

What is needed are apparatuses and methods for effectively storing and delivering particles, especially particles that are capable of interlocking. An improved apparatus would prevent particles from engaging one another such that they could become interlocked to inhibit flow from a syringe or tube. An effective solution would provide for both sterile packaging and precise delivery though a small incision.

### SUMMARY OF THE INVENTION

An embodiment of the invention is an apparatus for delivering a particle. The embodiment includes a fluid supply mechanism and a tube containing a particle. The tube is coupled to the fluid supply mechanism and has an opening through which fluid supplied from the fluid supply mechanism may pass from the inside of the tube to the outside of the tube. The tube may include a cap such that when the cap is removed from the tube, the shaped particle may pass from the tube with fluid from the fluid supply mechanism.

Another embodiment of the invention is a particle delivery apparatus with a tube and a plurality of shaped particles stacked in the tube such that the shaped particles are substantially co-linear along the longitudinal axis of the tube. The plurality of shaped particles includes particles that have a center portion and at least four extremities projecting from the center portion wherein the extremities provide interstitial spaces between adjacent extremities, each extremity having a base at the center portion, a distal end, and a length, wherein the interstitial spaces of a first of the particles will accept at least a portion of one extremity of a second of the particles that is adjacent to the first particle.

Another embodiment of the invention is a particle delivery apparatus with a first fluid supply mechanism, a second fluid supply mechanism, and a valve with a first port coupled to the first fluid supply mechanism, a second port coupled to the second fluid supply mechanism, and a third port, the valve being operable to selectively enable flow between at least any two of the ports.

Still another embodiment is a method of delivering a particle comprising the acts of filling a tube containing a shaped particle with a fluid such that the fluid is interspersed around the shaped particle, removing an enclosure from an end of the tube to open the end of the tube, and continuing to fill the tube with a fluid to force the shaped particle from the tube.

Yet another embodiment of the invention is a method of delivering one or more of a plurality of shaped particles that are stacked in a flexible tube substantially co-linearly along the longitudinal axis of the tube comprising the acts of applying a force to the tube to restrict the flow of shaped particles within the tube at a location that will enable a desired quantity of shaped particles to be forced from the tube, and forcing one or more of the shaped particles from the tube.

Another embodiment of the invention is a method of delivering one or more of a plurality of shaped particles that are stacked in a tube substantially co-linearly along the longitudinal axis of the tube comprising the acts of inserting a slideable plug into the tube and urging the plug toward the plurality of shaped particles to force one or more shaped particles from the tube.

Another embodiment of the invention is a method of delivering a particle to a wound comprising the acts of providing a first fluid supply mechanism, providing a second fluid supply mechanism, and providing a valve with a first port coupled to the first fluid supply mechanism, a second port coupled to the second fluid supply mechanism, and a third port, the valve being operable to selectively enable communication between at least any two of the ports. The valve is set to enable communication between the first port and the second port, and the contents of the first fluid supply mechanism and the second fluid supply mechanism are mixed. The valve is set to enable flow through the third port, and the mixed contents are delivered through the valve and into the wound.

### BRIEF DESCRIPTION OF THE DRAWINGS

FIG. 1 is a perspective view of an embodiment of a particle delivery apparatus.

FIG. 2 is a perspective view of an embodiment of a particle delivery apparatus.

FIG. 3 is a perspective view of shaped particles functional in a particle delivery apparatus.

FIG. 4 is a perspective view of a cap or enclosure for a particle delivery apparatus.

FIG. 5 is a perspective view of the coupling structures between a syringe and a tube of an embodiment of a particle delivery apparatus.

FIG. 6 is a perspective view of a particle delivery apparatus including a clip for restricting the flow of shaped particles within the apparatus.

FIG. 7 is a perspective view of a particle delivery apparatus including a tube that contains shaped particles and has an end through which particles are discharged that restricts the flow of particles.

FIG. 8 is a perspective view of an embodiment of a particle delivery apparatus with a valve and two fluid supply mechanisms.

FIG. 9 is a perspective view of an embodiment of a particle delivery apparatus with a slideable plug disposed within a tube containing shaped particles.

### DETAILED DESCRIPTION OF THE INVENTION

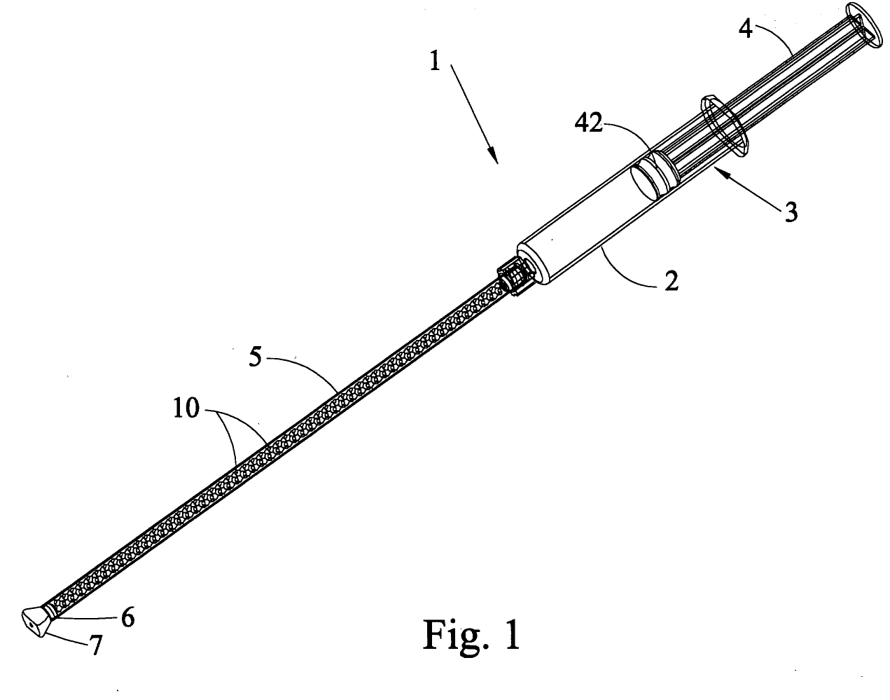

FIG. 1 shows a particle delivery apparatus 1. As illustrated, the particle delivery apparatus 1 includes a fluid supply mechanism 3, a tube 5 containing multiple shaped particles 10, and a cap 7 that is removable from one end of the tube 5. The tube 5 is coupled to the fluid supply mechanism 3. The particle delivery apparatus 1 may also include on opening between the inside of the tube 5 and the outside of the tube 5. Such an opening may be through any portion of the wall of the tube 5, but is particularly advantageous near a distal end 6 of the tube 5. The opening may be useful in allowing air and excess fluid from the fluid supply mechanism 3 to escape from the tube 5. The opening may be an opening 9 in the cap 7 as shown in FIG. 4 and described more fully below.

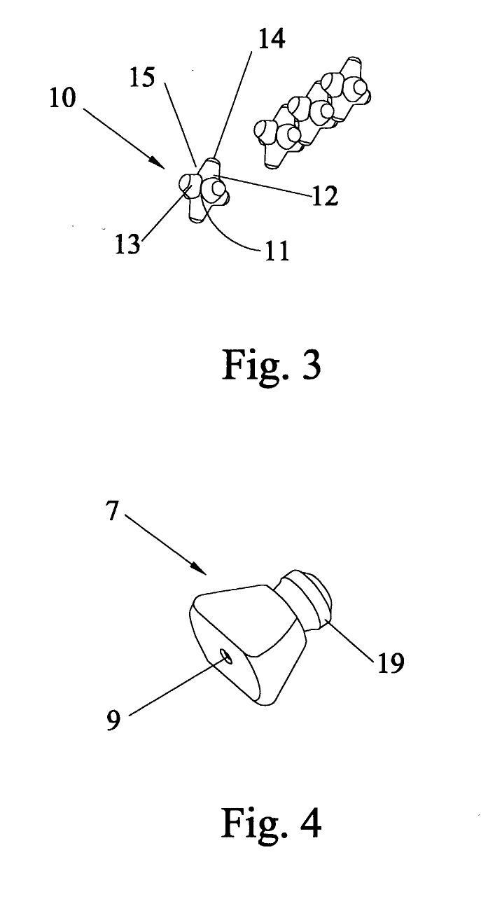

An enlarged view of the shaped particles 10 is illustrated in FIG. 3. Each shaped particle shown has a center portion 11, and six extremities 13 projecting from the center portion 1 1. Each of the six extremities 13 is substantially perpendicular to four other of the extremities. In some embodiments, the number of extremities 13 may be less than six. The extremities 13 provide interstitial spaces 15 between adjacent extremities 13. As depicted, each extremity 13 has a base 12 at the center portion 11, a distal end 14, and a length. As shown in FIG. 3, the interstitial spaces 15 of one of the shaped particles 10 will accept at least a portion of one extremity 13 of a second of the shaped particles 10 that is adjacent to the first particle. Many other features and embodiments of shaped particles are disclosed in the patent applications incorporate by reference above. All such features and embodiments are contemplated to fall within the claims presented herein, but the present invention is not limited to only those features and embodiments. Particles of various sizes and configurations would be operable and within claims of the invention.

The fluid supply mechanism 3 shown in FIG. 1 is a syringe. The syringe has a body 2 and a plunger 4. By pressing the plunger 4 down into the body 2, a pressure is developed in a fluid that is present in the body 2. When the fluid supply mechanism 3 is coupled to the tube 5, the pressure developed in the fluid supply mechanism 3 is transferred to the tube 5. The fluid supply mechanism 3 may be any type of mechanism that is capable of developing an adequate pressure. For example, the fluid supply mechanism 3 could be a hydraulic pump, pneumatic pressure source, or could be created by the compression of a containment structure containing a fluid. The body 2 and the majority of the plunger 4 as illustrated are made from a substantially transparent plastic. The sealing end 42 of plunger 4 as shown is made from an elastometric material such as synthetic or natural rubber. Alternate materials for each component would be sufficient and are known to one skilled in the art.

The fluid delivered from the fluid supply mechanism 3 may simply be a medium for carrying particles such as shaped particles 10, or it may provide other functional benefits. For example, the fluid may enhance the handling characteristics of the shaped particles and fluid mixture, or it may give the mixture biologically advantageous characteristics. Thorough explanations of many physical and biological benefits derived from the use of various substances that may be included in the fluid are given in the patent applications incorporate by reference above. In summary, possible substances and benefits include but are not limited to: substances that set or are non-setting by their nature or in response to time, temperature, or other stimuli, thus providing controllable physical characteristics; substances that are readily accepted by the human body; substances that aid in the agglomeration of the shaped particles; and substances that include biological agents such as antibiotics, growth factors, fibrin, bone morphogenetic factors, bone growth agents, chemotherapeutics, pain killers, bisphosphonates, strontium salt, fluoride salt, magnesium salt, sodium salt, or other substances that assist or enable the human body's healing processes. In one embodiment, the fluid is a gel comprised 88% (by weight) of high purity sterile water and 12% (by weight) of high purity glycerol and medical grade sodium carboxymethylcellulose.

The tube 5 may be substantially rigid or flexible, and may have a circular, rectangular, triangular, or other cross-sectional shape that provides containment for a shaped particle 10. A flexible tube 5 may be useful in reaching bone voids through a small incision and under computer or navigational guidance. A computer guided tube or its associated attachments may include a marker or guidance sensor, reflector, or transponder that could be tracked by an imaging or other tracking system and a computer. The tube 5 itself also serves as the sterile container in which shaped particles 10 may be transported to customers. The tube 5 may be a part of a kit that is contained within packaging that has been sterilized. For example, a kit could contain a tube 5 that has been filled with shaped particles 10, a fluid supply mechanism 3 such as a syringe, and a separate container of fluid to be used with the fluid supply mechanism. In other embodiments, the kit may not include either or both of the fluid supply mechanism and the separate container of fluid.

As illustrated in FIGS. 1 and 3, shaped particles 10 are stacked in a tube 5 such that the shaped particles 10 are substantially co-linear along the longitudinal axis of the tube 5. The longitudinal axis of the tube 5 illustrated is straight, but in the instance of a flexible tube, or a tube formed into a curve, the longitudinal axis of the tube may be curved. The tube 5 may also include graduations marked along the tube to indicate the quantity or volume of shaped particles that are present in the tube or that have been removed from the tube.

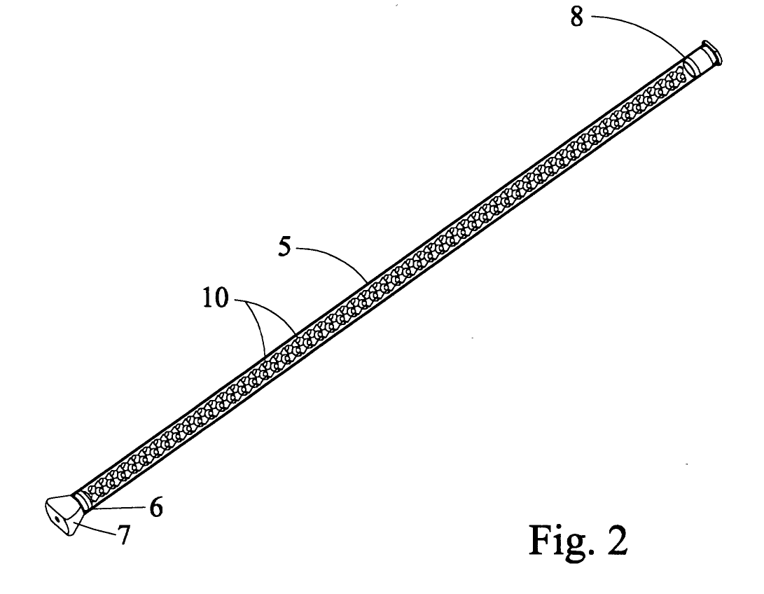

FIG. 2 shows an embodiment of the invention that does not include a fluid supply mechanism. In this embodiment, a separate fluid supply mechanism could be coupled to the tube 5 as described above. However, the invention would also be operable without a fluid supply mechanism by pouring shaped particles 10 from the tube 5 under the force of gravity. Pouring can be accomplished with or without fluid mixed with the shaped particles 10. In another embodiment, a ram, push-rod, or other such device that would fit within the inside diameter of the tube 5 may be used to force one or more of the shaped particles 10 from the tube 5. Alternatively, a sliding constriction applied along the outside of the tube 5 could be used to force out the shaped particles 10. The tube 5 illustrated is made of a clear plastic material, but any material providing adequate mechanical and chemical properties would be sufficient.

FIG. 4 depicts an enlarged view of the cap 7 that may be removably coupled to the tube 5. The cap 7 has an opening 9 through which fluid from the inside of the tube 5 may pass to the outside of the tube 5. For example, when fluid from a fluid supply mechanism is added to the tube 5 to be mixed with shaped particles 10, air would be forced from the tube 5 through opening 9. When the fluid from the fluid supply mechanism reaches the opening 9, it is also passed to the outside of the tube 5. Therefore, air and the fluid from the fluid supply mechanism, both fluids, pass from the inside to the outside of tube 5. A ridge 19 on the cap 7 provides for a compression fit of the cap 7 into the tube 5. However, with a reasonable amount of force, the cap 7 can be removed from the tube 5. With the cap 7 removed, the shaped particles 10 may pass from the tube 5 with fluid from a fluid supply mechanism.

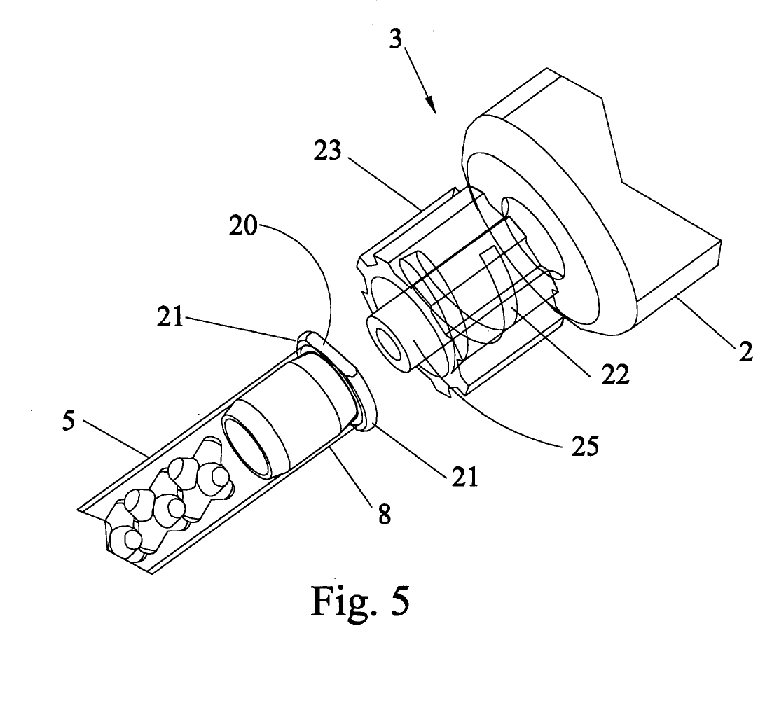

FIG. 5 illustrates the coupling between a first end 8 of tube 5 and the fluid supply mechanism 3. An attachment fitting 20 is press fit into the first end 8. The attachment fitting 20 has wings 21 that extend beyond the outside diameter of the tube 5. In some embodiments, the attachment fitting 20 may be attached to the tube 5 with an adhesive, or may be formed as an integral part of the tube 5. When the tube 5 is coupled to the fluid supply mechanism 3, the wings 21 engage threads 22 in an annular connector 23. The annular connector 23 is coupled to the body 2 of the fluid supply mechanism 3. By engaging the wings 21 with the threads 22, the first end 8 of tube 5 is coupled over a spout 25 of the fluid supply mechanism 3. The spout 25 fits tightly within the inside diameter of the attachment fitting 20 in the first end 8 of tube 5 and seals the coupling. The outside diameter of the spout 25 may be frusto-conical in shape such that further tightening of the wings 21 into the threads 22 causes an increasingly tight fit between the tube 5 and the spout 25.

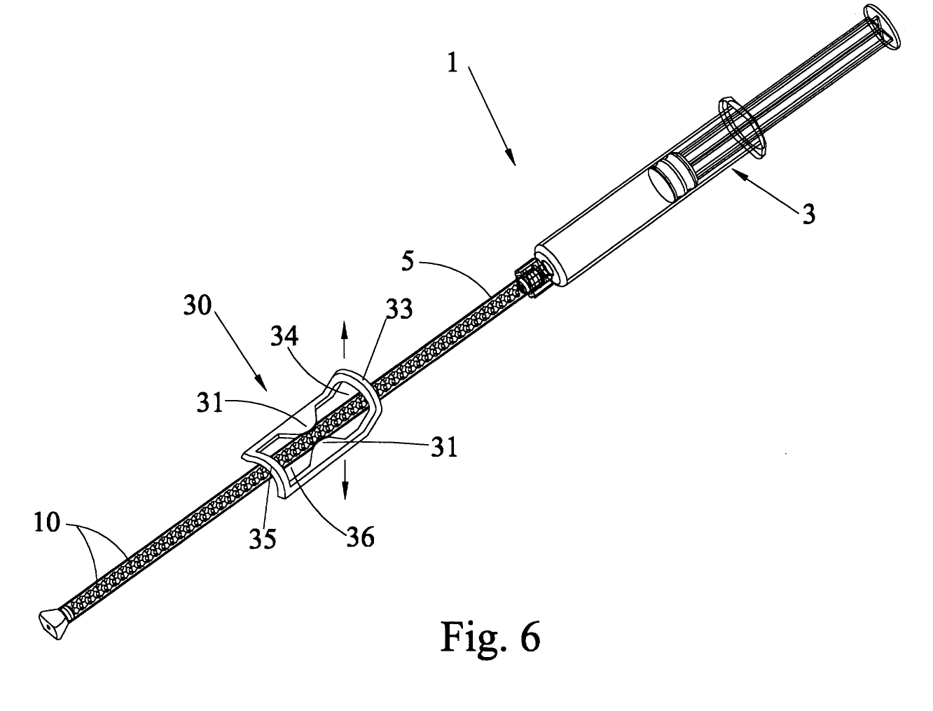

FIG. 6 shows an embodiment of the particle delivery apparatus 1 that includes a clip 30. The clip 30 functions by connecting onto tube 5 to restrict the flow of shaped particles 10 within the tube 5. The clip 30 illustrated has two opposing wings 31 with a gap between them that is less than the diameter of the tube 5. A cross member 35 connects the two sides of clip 30 on which the opposing wings 31 are mounted. The clip 30 has a raised end 33 that extends out of the plane which the remainder of the clip 30 generally occupies. The raised end 33 provides an opening 34 in the clip 30 that allows the clip 30 to be moved transverse (see arrows) to the longitudinal axis of the tube 5 while staying engaged about the tube 5 through a gap 36 between the cross member 35 and the opposing wings 31. By moving the clip 30 to a desired location along the tube 5 and sliding the clip 30 transverse to the longitudinal axis of the tube 5, the opposing wings 31 compress the tube 5 and restrict the flow of the shaped particles 10 within the tube 5. Restriction of the flow therefore only allows a desired amount of the shaped particles 10 to be released from the tube 5. By moving the clip 30 in an opposite, transverse direction, the opposing wings 31 become disengaged and the tube 5 slides freely through the opening 34 and the gap 36 of the clip 30. Any other clip or clamp that effectively restricts the flow of shaped particles 10 within the tube 5 would be adequate and is contemplated to be within the scope of the invention.

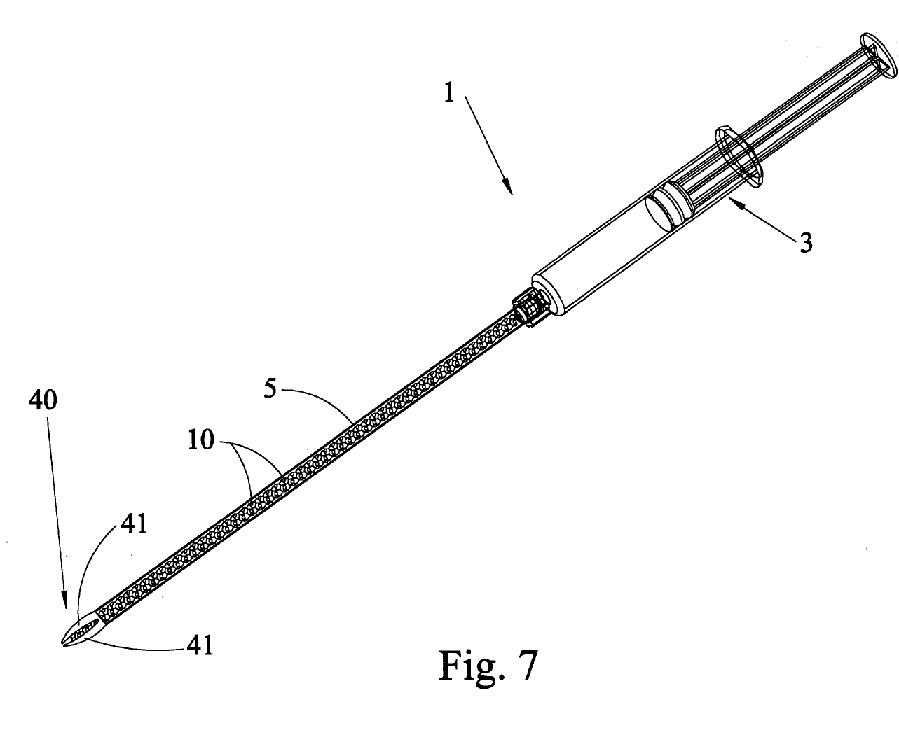

FIG. 7 illustrates a particle delivery apparatus 1 including a tube 5 that contains shaped particles 10 and has a restrictive end 40 through which particles 10 are discharged. The restrictive end 40 prevents shaped particles 10 from being discharged inadvertently or from falling out of the tube 5. However, under adequate force, shaped particles 10 may still be forced out of the tube 5 through restrictive end 40. In one embodiment, the restrictive end 40 has two or more fingers 41 that are elastically attached to the tube 5 such that by pushing a shaped particle 10 between them, the fingers 41 are spread apart. When the fingers 41 are spread apart, a shaped particle 10 may be passed between or among the fingers 41 and out of the restrictive end 40 if adequate force is applied. When the force is removed, the elasticity in the fingers draws the finger 41 back together and prevents the shaped particles 10 from passing from the tube 5. Other configurations for similarly restricting the flow of the shaped particle 10 would be evident to those skilled in the art and are within the scope of the present invention.

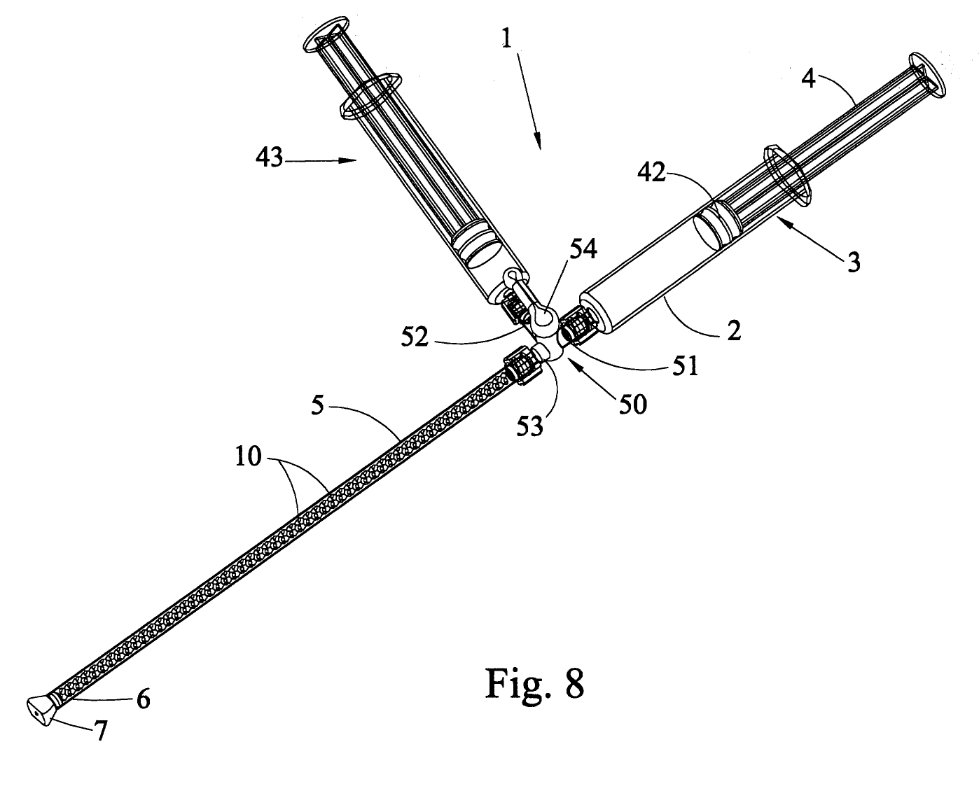

In FIG. 8, an embodiment of the invention that includes a valve 50 is shown. The valve 50 has a first port 51 coupled to the fluid supply mechanism 3, a second port 52 coupled to a second fluid supply mechanism 43, and a third port 53. The valve 50 also includes a stopcock 54 that is operable to selectively enable flow between at least any two of the ports 51, 52, 53. In some embodiments, flow may be enabled to pass from two of the ports simultaneously into a third. For example, flow may be enable through the first port 51 and the second port 52 simultaneously into the third port 53. As illustrated, a tube 5 containing shaped particles 10 is coupled to the third port 53. In this configuration and with the stopcock 54 enabling flow between the first port 51 and the third port 53, the device is operable as was described in association with FIG. 1 above.

In some embodiments, the fluid supply mechanism 3 contains one liquid and the second fluid supply mechanism 43 contains a second liquid. The liquids used may individually or in combination contain substances and provide therapeutic, physical, and other benefits as described above and in the patents incorporated by reference herein. In addition, one or both of the fluid supply mechanisms may contain particles that are small enough to be passed through the valve 50. Such particles may be mixed with liquids or other particles and may be supplied to the tube 5.

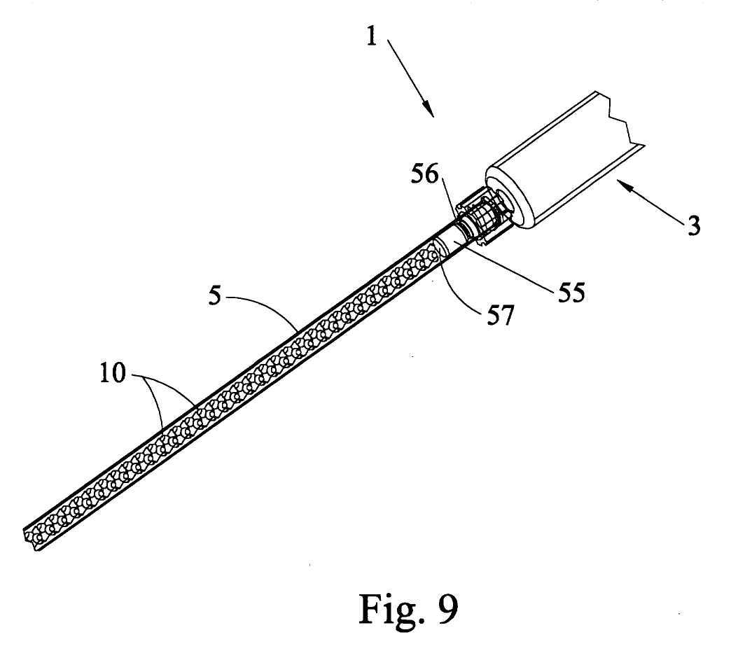

FIG. 9 depicts an embodiment of the invention with a slideable plug 55 disposed within a tube 5 containing shaped particles 10. The slideable plug 55 fits within the inside diameter of the tube 5. In some embodiments, the plug 55 forms a seal to prevent the contents on either side of the plug 55 from mixing with each other. The plug 55 can be urged by a force on its one side 56 toward the plurality of shaped particles 10 on its other side 57. Such a force can urge one or more of the shaped particles 10 out of the tube 5. The plug 55 may be used with a fluid supply mechanism, with a ram or push-rod, or in combination with a valve 50 (FIG. 8). The plug 55 may also serve as a sterile or non-sterile closure for the tube 5.

Compatible combinations of the embodiments illustrated in FIGS. 1, 2, and 6-9 are contemplated by the invention and are within the claims of the invention.

Methods of Use

In one method of use of the invention, one or more particles are delivered by filling a tube containing shaped particles with a fluid such that the fluid is interspersed around the shaped particles, removing an enclosure from an end of the tube to open the end of the tube, and continuing to fill the tube with a fluid to force the shaped particles from the tube. With specific reference to FIG. 1, the tube 5 may be filled with fluid from the fluid supply mechanism 3. The fluid is able to be interspersed around the shaped particles 10 because of the unique geometric shape of the shaped particles 10. If the particles were tablets, for example, the fluid would not readily come into contact with the abutting surfaces of the tablets. Similarly, if the particles were a densely packed powder, it would be difficult to cause the fluid to penetrate the powder. Due to an opening between the inside and outside of the tube 5, the fluid from the fluid supply mechanism is able to displace the air from the tube 5 and fill in among the shaped particles 10. Once the fluid is placed in the tube in a desired amount, the cap 7 is removed from the tube 5 to open the end of the tube 5. Continuing to supply fluid from the fluid supply mechanism 3 will force shaped particles 10 from the tube 5.

Another method of the invention includes delivering one or more of a plurality of particles that are stacked in a flexible tube substantially co-linearly along the longitudinal axis of the tube by applying a force to the tube to restrict the flow of shaped particles within the tube at a location that will enable a desired quantity of shaped particles to be forced from the tube, and forcing one or more of the shaped particles from the tube. Such a force may be applied by use of a clip 30, as illustrated in FIG. 6. The clip 30 could be used under the method to restrict the tube 5, and then the shaped particle 10 could be poured from the tube 5 under the force of gravity. Similarly, the tube 5 restricted by the clip 30 could have fluid supplied to the tube 5 to force the shaped particles 10 that are not restricted by the clip 30 from the tube 5. That is, the hydraulic force of the supplied fluid would not be significantly restricted by the clip 30, but some of the shaped particles 10 would be. Therefore, the fluid supplied would bypass the restricted shaped particles and force the unrestricted shaped particles from the tube 5. Similarly, application of the force under the method could be accomplished by a user grasping or pinching the tube 5 to restrict flow of the shaped particles 10 or by any other means of applying a force.

A method of delivering a particle to a wound under embodiments of the invention may be performed with the apparatus illustrated in FIG. 8. With the stopcock 54 of the valve 50 set to enable communication between the first port 51 and the second port 52, the contents of either fluid supply mechanism may be forced into the other fluid supply mechanism. The contents may be mixed by successively passing the contents from one fluid supply mechanism to the other. Alternatively, the contents may be mixed by passing the contents into one of the fluid supply mechanisms and shaking the fluid supply mechanism. Other methods of agitating the contents to achieve a desirable mixture are effective as well.

The contents that are mixed may be two similar or dissimilar fluids, or may include mixtures of particles that are small enough to be passed through the valve 50. Therefore, the resulting mixture may be either a fluid, a dry mixture of particles, or a fluid intermixed with particles.

Flow through the third port 53 is enabled by setting the valve 50. In the embodiment illustrated in FIG. 8, the stopcock 54 may be set to allow communication between either the first port 51 and the third port 53 or the second port 52 and the third port 53. In some embodiments, mixing and flow through the third port 53 can be accomplished by simultaneously forcing contents from fluid supply mechanism 3 and second fluid supply mechanism 43 through the valve 50 and through the third port 53.

The mixed contents from the fluid supply mechanism 3 and the second supply mechanism 43 may be suitable for delivery into a wound directly from the third port 53. In addition, it is advantageous in some clinical applications to apply an extension or other device to the third port 53 that better enables precise delivery to the wound site. As shown in FIG. 8, a tube 5 with shaped particles 10 is coupled to the third port 53. In delivery of the mixed contents to a wound, the contents may be forced into the tube 5 containing shaped particles 10 such that the contents are interspersed around the shaped particle 10. The contents and the shaped particles 10 are delivered to the wound by removing the cap 7 from the end of the tube 5 and continuing to fill the tube 5 to force the shaped particles 10 from the tube and into the wound. Delivery of the shaped particles 10 with embodiments of the invention having two fluid supply mechanisms may also be accomplished with apparatuses and methods describe in more detail herein in association with embodiments having a single fluid supply mechanism.

Another method of the invention includes delivering one or more of a plurality of shaped particles that are stacked in a tube substantially co-linearly along the longitudinal axis of the tube by inserting a slideable plug into the tube, and urging the plug toward the plurality of shaped particles to force one or more shaped particles from the tube. A plug 55 that would be advantageous in carrying out this embodiment of the invention is describe in association with FIG. 9.

In various embodiments, the open end of the tube 5 is inserted through an incision in a patient prior to forcing shaped particles 10 from the tube 5. With such a method, the shaped particles 10 can be placed very close to or directly into a wound site through a very small incision. Additionally, the tube 5 may be used to move or pack down shaped particles 10 into a desired location within an incision or other wound. The delivery apparatus may also be used to measure and/or mix a fluid with shaped particles. For instance, the mixture of fluid and shaped particles could be forced from the tube into a conventional dish or boat to be scooped into a wound site. Such a use might be advantageous if additional mixing or setting requirements were needed for a particular mixture.

Advantages of the Invention

The apparatuses and methods disclosed provide for the effective storage and delivery of particles that are capable of interlocking. By stacking particles as disclosed, the particles do not interlock with one another in such a way that they will become difficult to deliver prior to use. Furthermore, the invention as disclosed enables very accurate and minimally invasive delivery of the particles. Apparatuses and methods for sterilely packaging the particles are also disclosed. In some embodiments, the disclosed invention provides superior convenience in the mixing of a fluid with the particles. Rather than various containers of the prior art, the particles may be shipped and mixed with fluid in a single, ready-to-use device, and delivered to a wound site from that device. In alternate embodiments, the particles do not need to be mixed with a fluid to be delivered.

What is claimed is:

1. A particle delivery apparatus comprising:
a fluid supply mechanism;
a tube containing a particle, the tube coupled to the fluid supply mechanism;
an opening though which fluid supplied from the fluid supply mechanism may pass from the inside of the tube to the outside of the tube; and
a cap removably coupled to one end of the tube wherein when the cap is removed from the tube, the particle may pass from the tube with fluid from the fluid supply mechanism.

2. The apparatus of claim 1 wherein the particle has a center portion, and at least four extremities projecting from the center portion wherein the extremities provide interstitial spaces between adjacent extremities, each extremity having a base at the center portion, a distal end, and a length, wherein the interstitial spaces of a first of the particles will accept at least a portion of one extremity of a second of the particles that is adjacent to the first particle.

3. The apparatus of claim 1 wherein the particle has six extremities.

4. The apparatus of claim 3 wherein each of extremities is substantially perpendicular to four other of the extremities.

5. The apparatus of claim 1 wherein the fluid supply mechanism is a syringe.

6. The apparatus of claim 1 wherein the tube has a substantially circular cross-section.

7. The apparatus of claim 1 wherein the tube has a substantially rectangular cross-section.

8. The apparatus of claim 1 wherein the tube has a triangular cross-section.

9. The apparatus of claim 1 wherein the tube is flexible.

10. The apparatus of claim 1 wherein the opening is in the cap.

11. A particle delivery apparatus comprising:
a tube; and
a plurality of shaped particles stacked in the tube such that the shaped particles are substantially co-linear along the longitudinal axis of the tube;
wherein the plurality of shaped particles includes particles that have:
a center portion; and
at least four extremities projecting from the center portion wherein the extremities provide interstitial spaces between adjacent extremities, each extremity having a base at the center portion, a distal end, and a length, wherein the interstitial spaces of a first of the particles will accept at least a portion of one extremity of a second of the particles that is adjacent to the first particle.

12. The apparatus of claim 11 wherein the tube has a substantially circular cross-section.

13. The apparatus of claim 11 wherein the tube has a substantially rectangular cross-section.

14. The apparatus of claim 11 wherein the tube has a triangular cross-section.

15. The apparatus of claim 11 wherein the tube is a flexible tube.

16. The apparatus of claim 15 further comprising a clip releasably connectable to the tube to restrict the flow of shaped particles within the tube.

17. The apparatus of claim 11 wherein the longitudinal axis of the tube is curved.

18. The apparatus of claim 11 wherein the end of the tube through which particles are discharged restricts the flow of particles.

19. The apparatus of claim 11 wherein one or more of the plurality of shaped particles has six extremities.

20. The apparatus of claim 19 wherein each of extremities is substantially perpendicular to four other of the extremities.

21. The apparatus of claim 11 further comprising a plug disposed within the tube.

22. The apparatus of claim 21 wherein the plug is slideable within the tube.

23. The apparatus of claim 22 wherein the plug is urged by a force on its one side toward the plurality of shaped particles on its other side to force one or more shaped particles out of the tube.

24. The apparatus of claim 11 further comprising a fluid supply mechanism coupled to a first end of the tube.

25. The apparatus of claim 24 wherein the fluid supply mechanism is a syringe.

26. The apparatus of claim 24 further comprising an opening though which fluid supplied from the fluid supply mechanism may pass from the inside of the tube to the outside of the tube.

27. The apparatus of claim 24 further comprising a cap removably coupled to a second end of the tube, wherein the cap has an opening through which fluid supplied from the fluid supply mechanism may pass from the inside of the tube to the outside of the tube, and wherein when the cap is removed from the tube, one or more of the plurality of shaped particles may pass from the tube with fluid from the fluid supply mechanism.

28. The apparatus of claim 11 further comprising a ram slideable through the tube to force one or more of the plurality of shaped particle from the tube.

29. A particle delivery apparatus comprising:
a first fluid supply mechanism;
a second fluid supply mechanism; and
a valve with a first port coupled to the first fluid supply mechanism, a second port coupled to the second fluid supply mechanism, and a third port, the valve being operable to selectively enable flow between at least any two of the ports.

30. The particle delivery apparatus of claim 29 wherein a tube containing a shaped particle is coupled to the third port.

31. The particle delivery apparatus of claim 30 wherein the particle delivery apparatus includes an opening though which fluid supplied from the fluid supply mechanism may pass from the inside of the tube to the outside of the tube.

32. The particle delivery apparatus of claim 30 wherein the particle delivery apparatus includes a cap removably coupled to one end of the tube wherein when the cap is removed from the tube, the shaped particle may pass from the tube with fluid from the fluid supply mechanism.

33. The particle delivery apparatus of claim 32 wherein the cap includes an opening though which fluid supplied from the fluid supply mechanism may pass from the inside of the tube to the outside of the tube, and wherein when the cap is removed from the tube, the shaped particle may pass from the tube with fluid from one or both of the first fluid supply mechanism and the second fluid supply mechanism.

34. The particle delivery apparatus of claim 29 wherein the first fluid supply mechanism contains a first liquid and the second fluid supply mechanism contains a second liquid.

35. The particle delivery apparatus of claim 29 wherein the first fluid supply mechanism contains a first liquid and the second fluid supply mechanism contains particles that are small enough to be passed through the valve.

36. A method of delivering a particle comprising the acts of:
filling a tube containing a shaped particle with a fluid such that the fluid is interspersed around the shaped particle;
removing an enclosure from an end of the tube to open the end of the tube; and
continuing to fill the tube with a fluid to force the shaped particle from the tube.

37. The method of claim 36 further comprising the act of inserting the open end of the tube through an incision in a patient before forcing the shaped particle from the tube.

38. The method of claim 36 further comprising the act of pressing the open end of the tube against the shaped particle that was forced from the tube to move the shaped particle into a desired location.

39. A method of delivering one or more of a plurality of shaped particles that are stacked in a flexible tube substantially co-linearly along the longitudinal axis of the tube comprising the acts of:
applying a force to the tube to restrict the flow of shaped particles within the tube at a location that will enable a desired quantity of shaped particles to be forced from the tube; and
forcing one or more of the shaped particles from the tube.

40. The method of claim 39 wherein applying a force to the tube includes connecting a clip to the tube that reduces an inner dimension of the tube.

41. The method of claim 39 wherein forcing one or more of the shaped particles from the tube includes moving the tube such that the force of gravity forces the shaped particles from the tube.

42. The method of claim 39 wherein forcing one or more of the shaped particles from the tube includes supplying fluid to the tube to force the shaped particles from the tube with hydraulic force.

43. The method of claim 39 further comprising the act of inserting an end of the tube through an incision in a patient before forcing the one or more of the shaped particles from the tube.

44. The method of claim 39 further comprising the act of pressing an end of the tube against the one or more of the shaped particles that were forced from the tube to move the one or more of the shaped particles into a desired location.

45. A method of delivering one or more of a plurality of shaped particles that are stacked in a tube substantially co-linearly along the longitudinal axis of the tube comprising the acts of:
inserting a slideable plug into the tube; and
urging the plug toward the plurality of shaped particles to force one or more shaped particles from the tube.

46. The method of claim 45 further comprising the act of inserting an end of the tube through an incision in a patient before forcing the one or more of the shaped particles from the tube.

47. The method of claim 45 further comprising the act of pressing an end of the tube against the one or more of the shaped particles that were forced from the tube to move the one or more of the shaped particles into a desired location.

48. A method of delivering a particle to a wound comprising the acts of:
providing a first fluid supply mechanism;
providing a second fluid supply mechanism;
providing a valve with a first port coupled to the first fluid supply mechanism, a second port coupled to the second fluid supply mechanism, and a third port, the valve being operable to selectively enable communication between at least any two of the ports;
setting the valve to enable communication between the first port and the second port;
mixing the contents of the first fluid supply mechanism and the second fluid supply mechanism;
setting the valve to enable flow through the third port; and
delivering the mixed contents through the valve and into the wound.

49. The method of claim 48 wherein mixing the contents of the first fluid supply mechanism and the second fluid supply mechanism includes mixing fluid from the first fluid supply mechanism with particles from the second fluid supply mechanism, the particles being small enough to be passed through the valve.

50. The method of claim 49 wherein mixing the fluid and the particles includes forcing the fluid into the second fluid supply mechanism and agitating the particles and fluid.

51. The method of claim 50 wherein the agitating includes shaking the second fluid supply mechanism.

52. The method of claim 49 wherein mixing the fluid and the particles includes forcing the particles into the first fluid supply mechanism and agitating the particles and fluid.

53. The method of claim 52 wherein the agitating includes shaking the first fluid supply mechanism.

54. The method of claim 48 wherein mixing the contents of the first fluid supply mechanism and the second fluid supply mechanism includes mixing fluid from the first fluid supply mechanism with fluid from the second fluid supply mechanism.

55. The method of claim 48 wherein mixing the contents of the first fluid supply mechanism and the second fluid supply mechanism includes forcing the contents of the first fluid supply mechanism into the second fluid supply mechanism and then forcing the contents of the second fluid supply mechanism into the first fluid supply mechanism.

56. The method of claim 48 wherein setting the valve to enable flow through the third port includes setting the valve to communicate with either the first port or the second port.

57. The method of claim 48 wherein setting the valve to enable flow through the third port includes setting the valve to communicate with both the first port and the second port.

58. The method of claim 48 wherein delivering the mixed contents through the valve and into the wound includes delivering the mixed contents into a tube containing a shaped particle such that the contents are interspersed around the shaped particle.

59. The method of claim 58 wherein delivering the mixed contents through the valve and into the wound includes removing an enclosure from an end of the tube to open the end of the tube, and continuing to fill the tube to force the shaped particle from the tube.
